(19) United States
(12) Patent Application Publication
(10) Pub. No.: US 2025/0009439 A1
(43) Pub. Date: Jan. 9, 2025

(54) SYSTEM AND METHOD FOR SURGICAL STATE PREDICTION

(71) Applicant: Hoth Intelligence, Inc., Philadelphia, PA (US)

(72) Inventors: Tyler Alexander, Philadelphia, PA (US); Andrew Conklin, Philadelphia, PA (US); Jonathan Cohen, Philadelphia, PA (US); Remi Prince, Philadelphia, PA (US); Toryn Farr, Philadelphia, PA (US)

(73) Assignee: Hoth Intelligence, Inc., Philadelphia, PA (US)

(21) Appl. No.: 18/766,177

(22) Filed: Jul. 8, 2024

Related U.S. Application Data
(60) Provisional application No. 63/525,505, filed on Jul. 7, 2023.

(51) Int. Cl. A61B 34/20; A61B 34/10; A61B 34/30

(52) U.S. Cl. CPC A61B 34/20; A61B 34/10; A61B 34/30; A61B 2034/105; A61B 2034/107; A61B 2034/2059

(57) ABSTRACT

In variants, a method can include determining measurements of a surgical procedure, determining a set of feature states for elements depicted and not depicted within the measurements, tracking the set of features, determining a procedure step, predicting a set of future feature states based on the set of feature states and the procedure step, and performing further analyses based on the set of feature states and the set of predicted future feature states in order to facilitate measurement and improvement of a surgical procedure

### CROSS-REFERENCE TO RELATED APPLICATIONS

This application claims the benefit of U.S. Provisional Application No. 63/525,505, filed 7 Jul. 2023, which is incorporated in its entirety by this reference.

This application incorporates by reference U.S. application Ser. No. 17/719,043, filed 12 Apr. 2022, which is incorporated herein in its entirety by this reference.

### TECHNICAL FIELD

This invention relates generally to the surgical assistance field, and more specifically to a new and useful system and method for predicting a procedural state in the surgical assistance field.

### BRIEF DESCRIPTION OF THE FIGURES

FIG. 1 is a schematic representation of a variant of the method.

FIG. 2A-2C are schematic representations of variants of the method.

FIG. 3 is a schematic representation of a variant of data passed through the system.

FIG. 4 is an example of variants of measurement components and features.

FIG. 5A-5D are examples of variants of feature states.

FIG. 6 is an example of a variant of determining a set of feature states.

FIG. 7 is an example of a variant of S200 and S300.

FIGS. 8A and 8B are schematic representations of variants of S400.

FIGS. 9A and 9B are schematic representations of variants of state prediction model training.

FIG. 10 is a schematic representations of a variant of S500 and S600.

FIG. 11 is a schematic representation of a variant of a procedure state prediction model training and use.

### DETAILED DESCRIPTION

The following description of the embodiments of the invention is not intended to limit the invention to these embodiments, but rather to enable any person skilled in the art to make and use this invention.

### 1. Overview

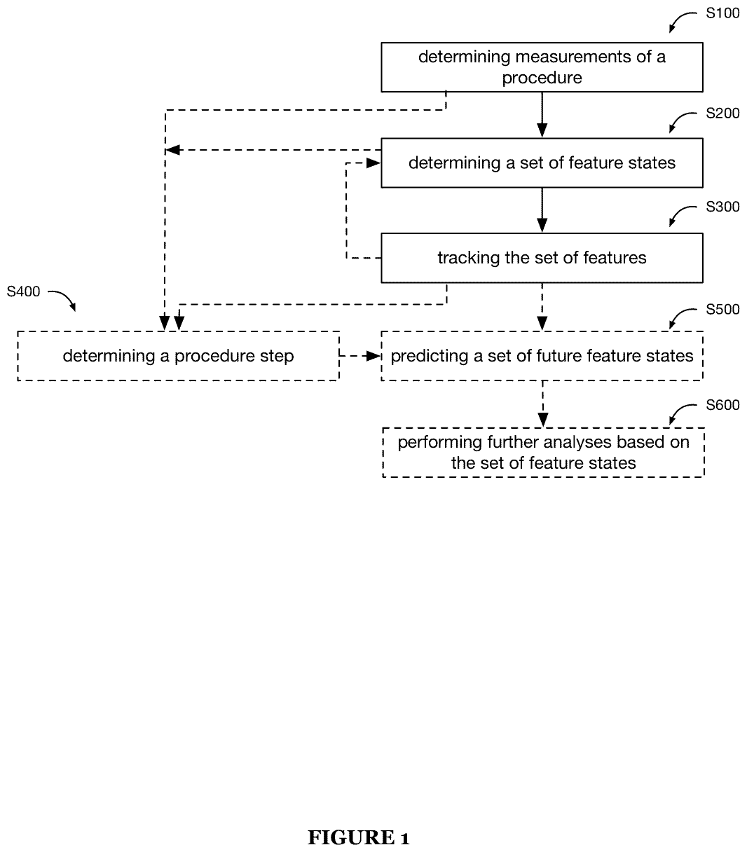

In variants, as shown in FIG. 1, a medical augmentation method can include: determining measurements of a procedure S100, determining a set of feature states S200 from the measurements, tracking the set of features S300, optionally determining a procedure step S400, optionally predicting a set of future feature states S500, optionally performing further analyses based on the set of feature states S600, and/or any other suitable steps.

In an illustrative example, during a medical procedure (e.g., surgical procedure), measurements of the procedure and subject 300 can be captured (e.g., in situ depth measurements, video measurements, etc.), a 3D representation of the subject 200 can be registered with the measurements 300, and the pose and trajectory of medical structures (features of interest; e.g., hands, instruments, etc.) used during the medical procedure can be extracted from the measurements 300 and tracked relative to one or more structures of the 3D representation 200. The 3D representation 200 can be constructed from cross-sectional subject scans (e.g., semantically-segmented CT scans), and can include a virtual model of an external subject layer (e.g., skin; bone, such as skull) and one or more virtual models of internal subject layers (e.g., soft tissues, organs, bones, etc.). The 3D representation 200 can be registered to the subject 20 depicted within the measurements 300 by: identifying keypoints in both the 3D representation's external layer and in the measurements 300; matching the keypoints (e.g., using ICP) between the external layer and the measurements 300; and aligning the 3D representation 200 to the subject measurements based on the keypoint matches. In variants, one or more layers of the 3D representation 200 (e.g., internal layers) can be overlaid over the measurements 300 (e.g., in an extended reality headset) based on the registration.

In this example, as the procedure progresses, the 3D representation 200 can remain registered (e.g., using the initial registration, iterative registration, etc.), wherein the state of each medical structure (e.g., pose, kinematics, trajectory, etc.) is extracted and tracked, relative to one or more layers of the 3D representation 200, using the registration. For example, the state of a medical structure (e.g., feature of interest, FOI) can be tracked by iteratively extracting the structure state in an augmented reality headset coordinate frame or measurement coordinate frame, transforming the structure state into the 3D representation coordinate frame based on the registration, determining a pose of the structure relative to an internal layer or structure, and tracking the structure pose relative to the internal layer over time. This can create a database of medical structure states relative to internal and/or external subject structures for one or more medical procedures.

In variants, the procedure can be temporally segmented into procedural steps (e.g., manually by the medical practitioner, using a procedure segmentation model, etc.), wherein the structure states can be stored in association with different procedural steps. In other variants, the structure tracking system can be used to determine whether and/or how closely a procedure is following a predetermined trajectory.

In variants, this database can be used in one or more ways. In a first example, historical structure state data can be used to determine a target trajectory or series of structure states for a procedure or step thereof (e.g., using averaging, etc.). In a second example, the historical structure state data can be used to train a trajectory prediction model to predict a structure trajectory or pose for the procedure or step thereof, given a prior structure state and/or internal structure information (e.g., pose relative to an external structure). The predicted or optimal trajectories (target trajectories) can be: displayed to the surgeon (e.g., within an AR display) to guide a surgical action, used to warn a surgeon when they are erring from the target trajectory, used to generate control instructions for a surgical robot, used to determine a “surgical score” representing the similarity between the actual trajectory and the target trajectory (e.g., during or after the procedure), or otherwise used.

However, the method can be otherwise performed.

### 2. Technical Advantages

Variants of the technology for predicting a procedural state can confer several benefits over conventional systems and methods.

First, typical medical procedures may be performed with a high degree of variability (e.g., in procedure and/or outcomes). While common outcome metrics include success rates and rates of complications such as infections, relatively little of what goes on during an individual procedure is recorded or analyzed. By capturing source measurements 300 of a medical procedure as it is performed, variants of the technology can determine how procedures are performed, and how medical providers 10 adapt to varying scenarios. By analyzing measurements 300 of many surgeries and determining an optimal tracked structure path for each step of a procedure, variants of the technology can additionally: train robots of varying degrees of freedom and varying end effector configurations to autonomously replicate a surgery; predict a timeseries of future procedure states; present a best surgical path; rate surgical performance; and/or otherwise improve the performance of a medical procedure. By tracking the trajectories of structures involved in a medical procedure over time, the system and method can enable analysis that leads to an improved understanding of the set of motions that characterize an effective procedure (e.g., decouples the data from the specific structural positions at each point in time). In addition, the trajectories describe movement patterns through space, and an analysis thereof can be more easily generalized across medical procedures with structures of variable characteristics (e.g., surgeon hand sizes, left and right-handed surgeons, varying body structures of subjects 20, etc.).

Second, variants of the method enable structure states (e.g., poses, kinematics, trajectories, etc.) to be tracked relative to the subject anatomy (e.g., internal subject structures). Because conventional methodologies only tracked structures relative to external subject features, not internal subject features, the instant technology creates a novel dataset that can be used to generate more accurate structure trajectory predictions, more accurate automated surgery control instructions, more accurate procedure outcome predictions, richer analyses, and/or otherwise used. In variants where the camera pose relative to the subject 20 is mobile (e.g., when the surgeon is wearing an extended-reality headset), this technology can enable continuous instrument tracking and/or guidance, even when external visual markers on the subject 20 cannot be seen due to the current pose of the camera, since the pose of the internal subject structures relative to the camera are still known.

Third, using both structure states observed from measurements 300 (observed feature states) and inferred structure states (inferred feature states) enables the system to gather higher-quality information about a procedure. Tracking inferred feature states enables the method to predict feature positions based on internal structures not seen by the camera (e.g., a tumor under the skin), non-visible portions of visible structures (e.g., the back side of an organ), surgical implements which cannot be seen by the camera (e.g., a stent traveling through an artery), and other non-visible features. Additionally, the method can include “remembering” feature states (e.g., feature poses) previously determined from observed features even when motion of the camera causes those observed features to be occluded.

Fourth, the method includes differentiating relevant feature states and measurements 300 from irrelevant feature states and measurements 300, which enables higher-quality data for training. Additionally, evaluating a set of measurements 300 or feature states as irrelevant can prevent erroneous feature state predictions based on unseen motion types during inference (e.g., a surgeon wiping their hands).

However, the technology can confer any other suitable benefits.

### 3. System

The system functions to identify and track structures involved in one or more medical procedures over time. The system can be used with a set of measurements 300, a set of features, a set of feature states, and/or other data.

The measurements 300 function to provide information about a medical procedure. The measurements 300 are preferably of the medical procedure, more preferably of the subject 20, the instruments, and optionally practitioner hands, but can additionally or alternatively include measurements 300 of the operating environment, and/or any other measurement. The measurements 300 are preferably sampled during the medical procedure, but can alternatively be simulated or otherwise obtained. The measurements 300 are preferably for a single procedure, but can alternatively be for a plurality of procedures. The measurements 300 preferably include a timeseries of measurements 300, but can alternatively include a single measurement.

The measurements 300 can depict one or more subjects 20, providers 10, medical devices, and/or any other entity Subjects 20 can include patients (e.g., human patients receiving a medical service), animals (e.g., veterinary subjects 20), and/or any other subject 20. Providers 10 can include physicians, surgeons, nurses, doctors, any other medical providers 10, medical robots, and/or any other person or entity performing an action.

Measurements 300 of the medical procedure can include imagery (e.g., RGB images), video, audio, depth measurements (e.g., point clouds, time of flight measurements, stereo images, depth maps, etc.), acceleration, LiDAR measurements 300, and/or other measurements 300. One or more measurements 300 of the same or different type can be contemporaneously obtained. Measurements 300 can be sampled using one or more sensors (e.g., a camera, a microphone, depth sensor, accelerometer, point cloud sensor, etc.). Sensors can be standalone (e.g., a mounted camera, a video recording system, etc.), incorporated into a device such as a mixed reality headset (e.g., an extended reality headset, a virtual reality (VR) headset, an augmented reality (e.g., AR) headset, XR/VR/AR glasses, etc.), incorporated into a portable device (e.g., a smartphone, a standalone camera, etc.), and/or otherwise configured. In examples, the system can include one or more cameras positioned: above a subject 20 during a procedure, adjacent to a subject 20 during a procedure, coupled to a provider 10 performing a procedure (e.g., a medical provider 10), mounted in the corner of a room (e.g., operating room) during a procedure, and/or otherwise positioned. In examples, the measurements 300 can be sampled from the provider's field of view or perspective. However, the camera can sample imagery of a procedure from any other suitable orientation. In variants with multiple sensors, sensors can be calibrated and registered relative to each other: before a procedure, during a procedure (e.g., iteratively), or not calibrated or registered.

In examples, the sensors can be paired with (e.g., mounted to) a display mechanism. The display mechanism can be a screen (e.g., an opaque screen, a transparent or translucent screen, an AR headset screen, a monitor, etc.); a projector (e.g., configured to project information onto a distal surface, such as onto the subject 20); a 3D display (e.g., configured to project one or more visual slices into a volumetric display); and/or another mechanism. The sensors can be mounted to the display mechanism (e.g., move with the display mechanism), be separate from the display mechanism (e.g., transmit measurements 300 to the display mechanism for display), and/or be otherwise associated with the display mechanism.

One or more distinct measurements 300 can each be associated with a timestep and/or any other metadata value. In examples, a timestep can reference a time elapsed since the start of a procedure, the start of a procedure step, and/or any other suitable reference time, can be a wall clock time (e.g., minutes, seconds, time of day, etc.), and/or can be otherwise defined. In examples, the timestep for a measurement, procedure step, or procedure can be determined automatically (e.g., when a measurement begins, when a new procedure step is determined, relative to the beginning of the procedure, etc.), and/or responsive to an input (e.g., an indication of a new step), manually performed, and/or otherwise performed. However, the measurements 300 can be otherwise configured.

The system can function to track one or more physical structures. Physical structures can be associated with a subject 20, a provider 10, a medical device, an object, and/or any other suitable organism and/or item. Physical structures associated with subjects 20 and/or providers 10 can be anatomical structures defined at varying levels of granularity (e.g., body, limb, digit, region, etc.).

Physical structures associated with a subject 20 (e.g., subject structures) can include: interior structures (e.g., anatomical structures such as: bones, organs, blood vessels, nerves, fat, abnormal growth, tumor, any body tissues, etc.), exterior structures (e.g., surface structures, head, face, abdomen, extremity, digit, skin, hair, eye, exterior abnormal growth, any region between air (or another medium) and an interior structure, etc.), visible structure, concealed structure (e.g., not depicted in a measurement, etc.), partially-concealed structure, and/or any other physical structure of a subject 20.

Physical structures associated with a provider 10 (e.g., provider structures) can include: a hand (e.g., left hand, right hand, etc.), a finger (e.g., index finger, middle finger, ring finger, pinkie finger, etc.), a thumb, an arm, a leg, a joint (e.g., of a finger, thumb, arm, leg, etc.), a bone, an eye, a back, a neck, any exterior structure, any interior structure, medical instruments and/or any other feature and/or portion thereof, of a provider 10 and/or a medical instrument.

Features associated with a medical instrument can include the medical instrument itself and/or a component thereof (e.g., proximal end, distal end, body, visual identifier, QR code, etc.). In a first example, a physical structure associated with a provider 10 is a set of fingers used to manipulate subject anatomy. In a second example, a physical structure associated with a provider 10 is a medical instrument used to modify subject anatomy. However, physical structures can be otherwise defined.

The 3D representation 200 (e.g., a “virtual patient model”) is preferably a virtual representation of the subject 20, more preferably a virtual representation of one or more subject structures, but can additionally or alternatively represent other entities. In variants, the 3D representation 200 can be the “patient-specific image reconstructed model” “3D representation” and/or “3D reconstruction” disclosed in U.S. application Ser. No. 17/719,043, filed 12 Apr. 2022, incorporated herein in its entirety by this reference, or be any other model. The 3D representation 200 can include one or more virtual structures. The virtual structures can be referenced to or share a common reference frame (e.g., generated from the same set of subject scans), registered, and/or otherwise aligned.

The virtual structures can represent: subject layers (e.g., patient layers), subject structures (e.g., leg, arm, head, etc.), and/or other portions of subject anatomy. Subject layers can include external layers, internal layers, and/or other layers. External layers can include skin, bone, and/or other external layers. External layers preferably have features that are detectable from outside the subject 20, such as geometric features (e.g., keypoints, etc.) or visual features, but can be otherwise defined. The external layers are preferably static (e.g., does not move relative to another portion of the external layer, but can alternatively be actuatable. Internal layers (e.g., an “internal model”) can include organs (e.g., brain, kidneys, tumor, etc.), bones, and/or other internal subject structures. The layers can be nested, adjacent, or otherwise arranged.

In a first example, a set of virtual structures (e.g., “virtual external patient structure”) can represent external layers (e.g., an external model which includes external features). In a second example, a set of virtual structures (e.g., a “virtual internal patient structure”) can represent internal layers (e.g., an internal model which includes internal features).

Virtual structures can be represented by a 3D set of points (e.g., a point cloud), a wireframe, a surface, a mesh (e.g., convex hull), a mask, a depth map, and/or any other suitable type of representation to make up virtual structures. In an embodiment, the system can include multiple 3D representations 200, wherein each 3D representation 200 includes a different type of anatomical subject structure (e.g., bone, organs, blood vessels, etc.). In this embodiment, virtual structures within different or the same 3D representations 200 can be aligned with each other based on known relationships between features of each (e.g., the spinal cord is known to be within the spinal canal, etc.).

The pose and/or other characteristics of a subject structure represented by a virtual structure can be inferred based on the states of the virtual structure. For example, the subject structure characteristics can be determined based on observed relationships between measurement features (e.g., extracted from the measurement) and 3D representation features (e.g., of the internal structure and/or external structure within the 3D representation) In a first example, the pose of a target subject structure associated with a virtual structure can be determined based on the states of a set of observed features of the target subject structure (e.g., where part or all of the target subject structure is visible). In a second example, the pose of the subject structure associated with a virtual structure can be determined based on a set of observed features of other subject structures with a known relative pose relative to the virtual structure associated with the subject structure.

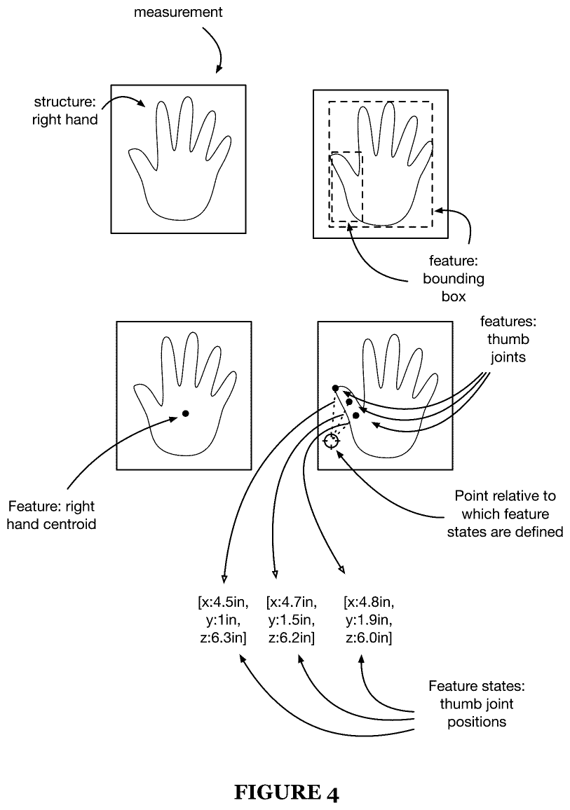

The system can function to track a physical structure by tracking one or more features associated with the structure (e.g., a subject 20, a provider 10, a medical device, and/or any other target) relative to another structure (e.g., another physical structure, a virtual structure, etc.) and/or features thereof. Features function as trackable elements within the scene (e.g., example shown in FIG. 4). Features can be associated with a subject 20 (e.g., “subject features”) and/or not be associated with a subject 20 (e.g., “non-subject features” or “medical tool features”), as in features associated with a provider 10, a provider's 10 tools, and/or the environment of the subject 20. Features can be associated with physical structures or virtual structures. Features can include a set of one or more points (e.g., spatial vector coordinate values), keypoints, segments, pixels, gradients, edges, blobs or pixel groups (e.g., locally unique blobs), lines (e.g., a line between other features), shapes, objects of a predetermined class (e.g., identified using a bounding box), object segment, 2-D regions, 3-D regions, surfaces, attributes (e.g., distances, maxima, minima, etc.), detections (e.g., structure instance detected in a measurement by an object detector), embeddings (e.g., vectors of numbers representing the regions of the measurement depicting the structures, determined using an encoder, etc.), and/or any other feature corresponding to a structure. Features can define a point on a structure (e.g., a center point, a centroid, an extremity, etc.), a set of points on a structure, a region encompassing a structure, one or more edges of the structure, and/or any other components of a structure. Additionally or alternatively, features can be associated with a physical or virtual marker placed on a structure. Each structure can be associated with one or more features. In an example, a structure (e.g., a hand, a head, etc.) is associated with a single feature (e.g., a centroid, a bounding box, etc.). In a second example, a structure (e.g., a hand, a head, etc.), is associated with a set of features which can include a set of points (e.g., a set of points defining the positions of each finger joint and/or fingertip of the hand, a set of points corresponding to pose of the head, etc.). In a third example, a structure (e.g., a hand, a head, etc.), is associated with a set of features which can include a set of lines (e.g., lines representing each rigid member of the hand, a set of lines corresponding to pose of the head, etc.). In a fourth example, a provider structure (e.g., a medical instrument) is associated with features which can include a visual marker (e.g., a QR code, etc.) which can be recognized by the method and used to determine the pose of the structure and/or a feature. Alternatively, features can include elements not associated with structures (e.g., audio features from an audio recording, pixels, signal features, semantic categories, etc.). Features can additionally be associated with feature characteristics (e.g., curvature, sharpness, semantic labels, etc.), kinematics (e.g., pose, velocity acceleration, trajectory, etc.), and/or other feature information. Features can be observed features (e.g., features of physical structures observed in a set of measurements 300) and/or virtual features (e.g., features inferred to exist based on a 3D representation of a subject 200 including virtual structures, wherein the virtual structures include one or more virtual features). Observed features and virtual features can overlap (e.g., when a feature in the 3D representation of the subject 200 is observed in a measurement) or not overlap. In examples, features can correspond to features of a subject's anatomy (e.g., “patient features”), and/or can correspond to features of provider structures (e.g., “non-patient structures”, “medical tool features” and/or “structure features”). However, features can be otherwise defined.

The system can function to track the state of a structure (e.g., a provider structure). Tracking the state of a structure can optionally include tracking the state of one or more features associated with the structure. The feature state functions to define a relationship between a feature and its environment. Feature states can be determined in S200 but can alternatively be determined at any other point. The feature state can include a set of values (e.g., numeric values, semantic values, etc.) describing a set of attributes and/or characteristics of the structure and/or features thereof, including: the position (e.g., a set of spatial vector coordinate values (x, y, z) defining the position), orientation, pose (e.g., position and orientation, etc.), trajectory (e.g., instantaneous trajectory), kinematics (e.g., velocity, acceleration, etc.), size, characterization or classification (e.g., “clean,” “broken,” “hemorrhaging,” “full,” etc.), quantity, operation state (e.g., “open,” “closed,” “off,” “on,” “injecting,” etc.), kinematics (e.g., pose, velocity acceleration, trajectory, etc.), procedural step, and/or any other suitable attribute of the structure and/or of features associated with the structure. The feature state can include the individual states, absolute states, relative states (e.g., relative positions, relative poses, distances therebetween, etc.), and/or aggregate states (e.g., quantity, average state values, etc.) of one or more features. Feature state values can be defined: relative to other features (e.g., observed features of external anatomy, virtual features within the 3D representation 200, provider features, etc.), relative to other structures (e.g., physical structures, virtual structures, subject structures, provider structures, etc.) relative to an origin of a coordinate system (e.g., a coordinate system defined by a set of points in space, defined by features of a structure detected within a set of measurements 300, defined as a synthetic coordinate system, etc.), relative to a measurement (e.g., as a pixel coordinate), and/or defined relative to any other point or set of points. Feature state values can be defined in 2D space (e.g., within the measurement) and/or 3D space (within the 3D representation 200; within the real world, etc.). A coordinate system can serve as a reference frame for feature states. In a first variant, feature state values can be defined within a coordinate system defined by the 3D representation 200 and/or structures therein (e.g., soft tissues). In a second variant, feature state values can be defined within a coordinate system defined by a set of feature states (e.g., of other features). However, the coordinate system can be otherwise defined. In a first specific example, a feature state can include a pose and/or trajectory of an observed feature, corresponding to a provider structure, relative to a virtual feature corresponding to a virtual structure representing a subject structure (e.g., track the feature state relative to a virtual internal patient structure represented by the 3D representation). In a second specific example, a feature state can include a pose and/or trajectory of an observed feature corresponding to a provider structure relative to an observed feature corresponding to a subject structure (e.g., track the feature state relative to global coordinates or an external patient structure, etc.). In examples, feature state values can be represented as a vector, matrix, virtual structure, and/or can be otherwise represented. In a specific example, a feature state is an encoding determined from information about a feature. However, feature state can be otherwise defined.

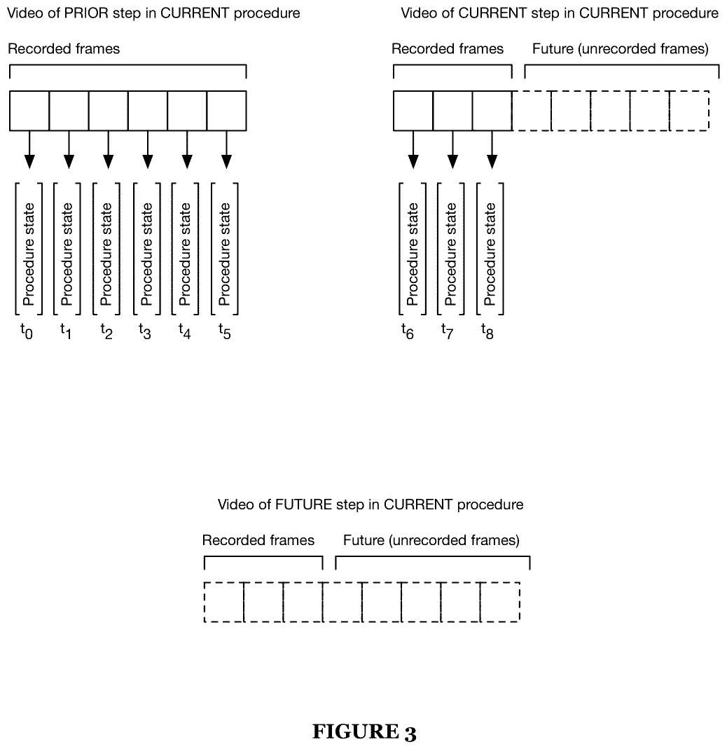

A procedure state can include a set of feature states at a given time step. The given time can be a point in time, a measurement (e.g., a frame), a procedure step, a time range, and/or any other suitable timestep. The procedure state can include the state of one or more features (e.g., see example in FIG. 3). Specific examples of information represented within a procedure state (e.g., by the set of values) can include: the relative configuration of each of a set of features depicted and/or not depicted within a measurement (e.g., at a given time point); the trajectories (e.g., instantaneous trajectories, relative trajectories, etc.) of each of a set of features depicted and/or not depicted within a measurement (e.g., at a given time point); the spatial vector coordinate values (x, y, z) defining each feature detected within a measurement and optionally a corresponding timestamp (t); and/or any other suitable set of state values. A procedure state can include the feature state for all features within a scene or a feature state for a subset of features within a scene. The subset of features within a scene can be determined based on relevance to the present procedure, proximity to a structure (e.g., a provider's surgical implement, association with a selected list of structures or structure types, trajectory, kinematics, and/or any other suitable feature attribute. However, the procedure state can be otherwise defined.

The state (e.g., procedural state, feature state, etc.) and/or state values can be associated with a procedure, a procedure step, a timestep (e.g., of the measurement(s) that the feature values were extracted from), and/or any other suitable information. State values can optionally include and/or be expressed relative to a corresponding timestep (t). However, states can be otherwise defined.

The system can additionally or alternatively function to segment a procedure into a series of steps. The steps are preferably serial, but can alternatively run in parallel. The system can optionally also assign measurements 300, features, and/or other information to different procedure steps. The procedure can be segmented: manually (e.g., a provider 10 marks the start and/or end of a step; an annotator marks the duration of a step after recording; etc.); automatically (e.g., inferred based on the procedure state matching a start, end, or intermediary process within a step; inferred based on auxiliary information, such as step start or stop indicators detected in a synchronized audio stream; segmented using a step classification model 110; etc.); and/or otherwise determined.

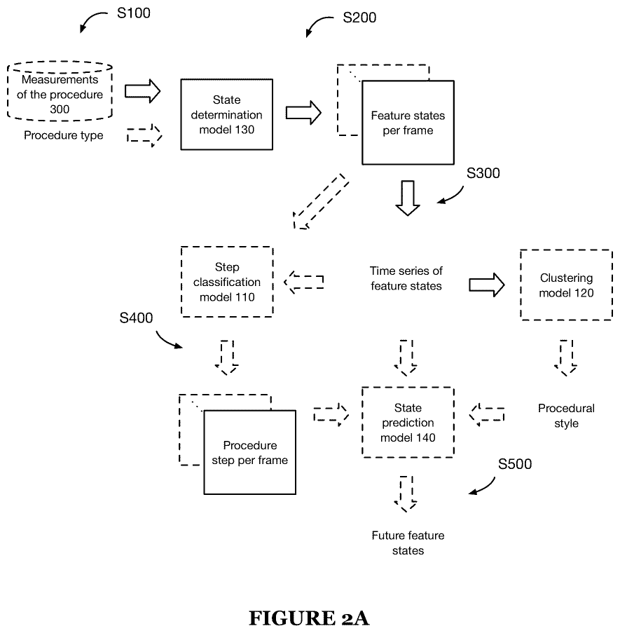

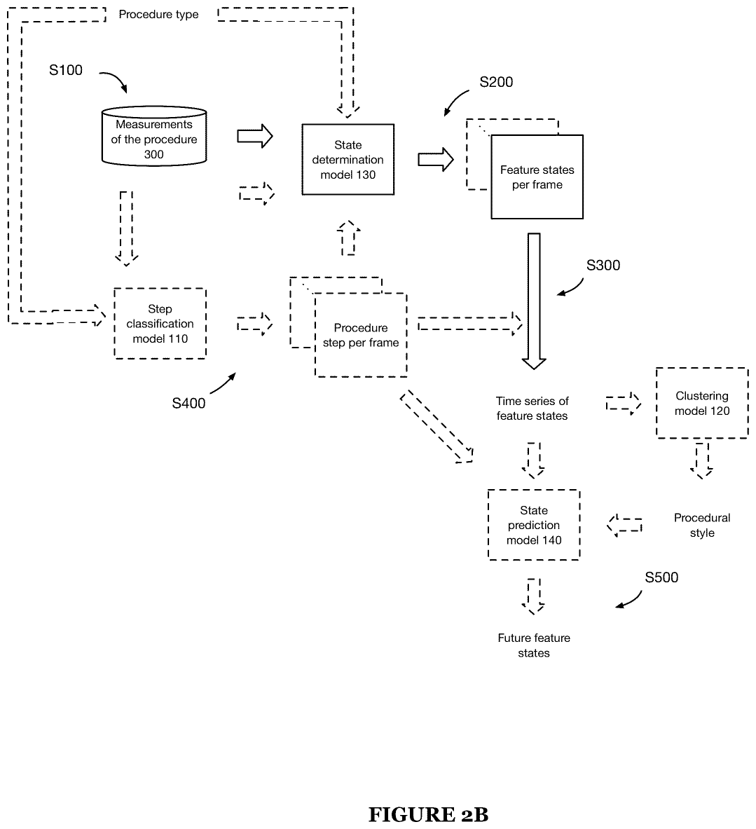

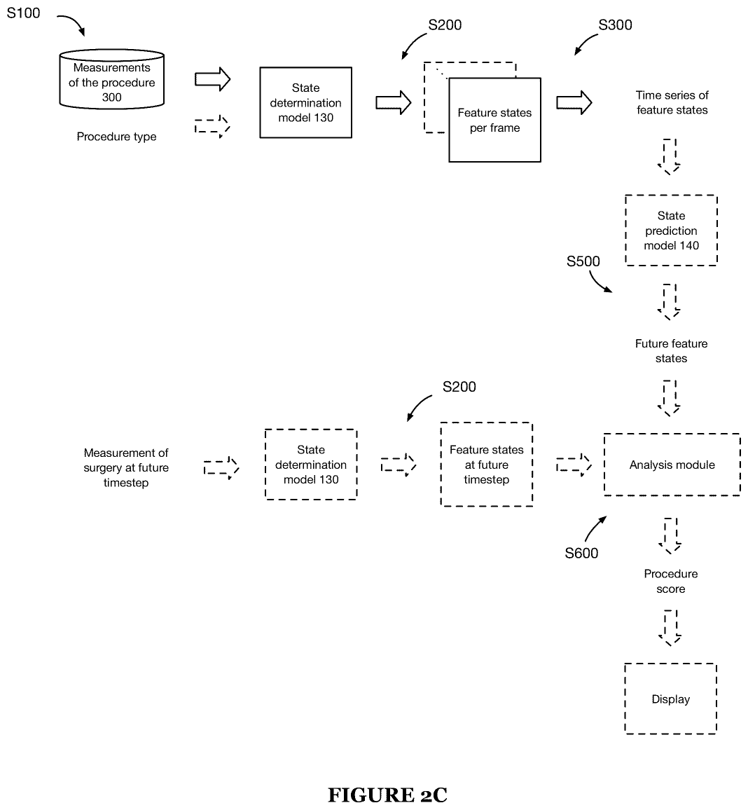

As shown in FIGS. 2A-C, the system can include one or more: step classification models 110, clustering models 120, state determination models 130, state prediction models 140, and/or any other suitable models and/or algorithms. Each model can be specific to a step, specific to a procedure, specific to a structure, specific to a set of features, specific to a provider implement (e.g., a surgical tool, a hand, etc.), specific to a subject 20, specific to a demographic, and/or specific to any other suitable procedure attribute. Alternatively models can be generic across one or more steps and/or procedures. The models can include classical or traditional approaches, machine learning approaches, and/or be otherwise configured. The models can include regression (e.g., linear regression, non-linear regression, logistic regression, etc.), decision tree, LSA, clustering, association rules, dimensionality reduction (e.g., PCA, t-SNE, LDA, etc.), neural networks (e.g., CNN, DNN, CAN, LSTM, RNN, encoders, decoders, deep learning models, transformers, etc.), ensemble methods, optimization methods, classification, rules, heuristics, equations (e.g., weighted equations, etc.), selection (e.g., from a library), regularization methods (e.g., ridge regression), Bayesian methods (e.g., Naive Bayes, Markov), instance-based methods (e.g., nearest neighbor), kernel methods, support vectors (e.g., SVM, SVC, etc.), statistical methods (e.g., probability), comparison methods (e.g., matching, distance metrics, thresholds, etc.), deterministics, genetic programs, hidden Markov models, and/or any other suitable model. The models can include (e.g., be constructed using) a set of input layers, output layers, and hidden layers (e.g., connected in series, such as in a feed forward network; connected with a feedback loop between the output and the input, such as in a recurrent neural network; etc.; wherein the layer weights and/or connections can be learned through training); a set of connected convolution layers (e.g., in a CNN); a set of self-attention layers; and/or have any other suitable architecture.

Models can be trained, learned, fit, predetermined, and/or can be otherwise determined. The models can be trained or learned using: supervised learning, unsupervised learning, self-supervised learning, semi-supervised learning (e.g., positive-unlabeled learning), reinforcement learning, transfer learning, Bayesian optimization, fitting, interpolation and/or approximation (e.g., using gaussian processes), backpropagation, and/or otherwise generated. The models can be learned or trained on: labeled data (e.g., data labeled with the target label), unlabeled data, positive training sets (e.g., a set of data with true positive labels, negative training sets (e.g., a set of data with true negative labels), and/or any other suitable set of data.

The classification model, clustering model 120, state determination model 130, the state prediction model 140, trajectory model, and/or any other suitable model can be trained using a sets of training procedure data representing training procedures (e.g., measurements 300, features, procedure states, feature states, and/or other information associated with a procedure).

The models can be trained and/or refined using the training procedure data. In an example, a model is trained to perform a particular operation and is subsequently refined based on training procedure data associated with a particular provider 10 or set of providers 10. Training procedure data can represent prior iterations of the procedure being performed (e.g., based on videos of surgeries), can represent prior iterations of other procedures being performed (e.g., for a model used to predict a particular step shared by multiple procedures), can represent virtual walkthroughs of the procedure being performed, and/or represent any other suitable form of procedure. Training procedure data can represent a procedure including multiple steps and/or can represent a single step or step type. Training procedure data can include metadata about the subject 20 and/or procedure (e.g. surgery time, surgery location, provider identification information, subject demographics, subject health constraints, surgical constraints, and/or any other suitable form of metadata). Training procedure data can be captured virtually or by a set of sensors. In a variant where training procedure data is captured virtually, training procedure data can be captured by recording virtual measurements 300 of a virtual operation (e.g., using a virtual camera) performed on a 3D representation of a subject 200. In a variant where training procedure data is captured by a set of sensors, the set of sensors can include any of the sensors used in S100 and/or by any other suitable sensor or set of sensors. Training procedure data can be sampled (e.g., by recording video), received (e.g., sampled by a device external to the system), calculated (e.g., using other models than the model for which training procedure data is being determined), and/or otherwise determined. Training procedure data can represent procedures performed by a particular provider 10 and/or different providers 10. Training procedure data can be labeled manually, labeled automatically (e.g., by a different model; for example, a step label generated by the classification model can be used as a training input for the state prediction model 140), and/or otherwise labeled and/or modified. The training procedure data can be filtered, labeled, and/or otherwise organized. The training procedure data can be organized according to a respective a cluster, a step, a step methodology, a procedure outcome, and/or any other metadata.

Training procedure data can optionally be segmented. Segmenting training procedure data can segment training procedure data based on whether the training procedure data is represents a particular step, whether the training procedure data is relevant to the procedure (e.g., filtering out training procedure data as being associated with an irrelevant action, like a provider 10 changing gloves or pausing, etc.), whether the training procedure data represents a particular procedure, whether the training procedure data represents a particular demographic, and/or based on any other suitable segmentation condition. Segmenting training procedure data can be performed manually, can be performed automatically based on supplementary data associated with the training procedure data (e.g., an audio recording describing the steps being performed), can be performed using a generic frame classification model, and/or can be otherwise performed. In an example, measurements 300 collected for a procedure (e.g., a video clip) can be separated into separate training procedure data sets for each step of the procedure (e.g., a set of per-step video clips; example shown in FIG. 2). In a second example, measurements 300 collected for multiple procedures with overlapping step types can be segmented by step type (e.g., determined using a step classifier trained on labeled step video clips, etc.), and the overlapping steps between procedures can be included in the same training procedure data set. In a third example, training procedure data can be stored in a database based on whether it is relevant to a procedure step associated with the database. However, training procedure data can otherwise be segmented.

The optional step classification model 110 functions to determine a step (e.g., a procedural phase, movement, movement pattern, etc.) of a procedure. The procedure step can be associated with a specific procedure, or be common across multiple procedures (e.g., suturing). In variants, a step classification model 110 can classify multiple steps (e.g., a multi-headed model). The input to the step classification model 110 can include measurements 300, features, feature states (e.g., all feature states for a given procedure state or a subset of feature states for the given procedure state; feature states for prior procedure states; a time series of feature states, feature tracks, etc.), current procedure steps, current procedure type, information about the subject 20, and/or any other suitable piece of information. In variants where the inputs to the step classification model 110 include feature states, the feature states can be subject feature states, provider feature states, and/or another suitable type of feature state. Additionally or alternatively, inputs to the step classification model 110 can be passed to multiple step classification models 110, each of which are classifiers specific to a particular step. In variants, the step classification model 110 can be or include a computer vision model, and/or any other suitable model. In a first variant, the step classification model 110 determines the procedure step based on a timeseries of states (e.g., procedure states, feature states, etc.) extracted from the set of measurements 300, or based on the set of measurements 300 directly (e.g., using a classifier, a pattern matching model, etc.). In a second variant, the step classification model 110 determines the procedure step using a set of heuristics. In an example, when each procedure is associated with a series of known steps, the current step is assumed to be the next uncompleted step in the series. In a second example, each procedure step is associated with a set of surgical implements, and the step is assumed to be the step corresponding to the surgical implements in the measurements 300. In a third variant, the step classification model 110 determines the procedure step based on an encoding (e.g., of the measurements 300, procedure state, etc.) determined for each procedure step.

Training the step classification model 110 can include using labeled, optionally standardized training procedure data containing a set of measurements 300, an optional set of input features, and an optional set of input feature states as training inputs. The set of measurements 300 can include a single measurement, measurements 300 spanning a particular time window, a particular number of measurements, and/or can be otherwise organized. The optional set of input features can include any suitable type of feature determined from the measurement. In an example, the set of measurements 300 includes a set of frames depicting a procedure step and the set of features includes a set of feature states determined based on the set of measurements 300. The optional set of input feature states can include outputs of the state determination model 130 (e.g., feature trajectories, etc.). Training the step classification model 110 can include using step labels and/or any other suitable information as classification targets. Step labels can be manually or automatically determined. In an example, step labels are determined via a manual labeling process. Training the step classification model 110 can include predicting a step label, comparing it to the classification target, and optionally updating the model (e.g., using backpropagation, etc.). However, the step classification model 110 can be otherwise trained.

However, the step classification model 110 can be otherwise configured.

The optional clustering model 120 (e.g., example shown in FIG. 2B) functions to determine a cluster for the procedural style of the procedure and/or procedure step being performed. This can be particularly useful when a procedure and/or step can be performed many different ways. In examples, clusters can include: dominant hand of the provider 10, a surgical style, and/or any other category affecting the way that a procedure is performed. The clustering model 120 can use: similarity measure-based clustering, k-means clustering, multi-layer clustering, and/or any other suitable clustering method. The clustering model 120 preferably ingests a time series of feature states for each of a set of features, a set of feature states, and/or any other suitable input. The clustering model 120 preferably outputs a cluster label, references to a set of inputs which are within a clusters, and/or any other suitable output. However, the clustering model 120 can be otherwise defined.

The state determination model 130 functions to determine the procedure state (e.g., a set of feature states) based on the measurements 300 and optionally prior measurements 300, procedure states for the prior measurements 300, a time series of feature states, and/or step labels for the corresponding measurements 300 or prior measurements 300. The state determination model 130 can be specific to a particular procedure step or can work on measurements 300 capturing multiple or all procedure steps. The output of the state determination model 130 preferably includes feature states (e.g., kinematic state) but can include any other suitable output, including object detections, confidence, and/or any other output. In a first variant, the state determination model 130 determines feature states based on measurements 300 alone. In a second variant, the state determination model 130 determines feature states based on the measurements 300 and a set of input features. In a third variant, the state determination model 130 determines feature states based on a set of features alone. However, the state determination model 130 can otherwise determine feature states. In examples, the state determination model 130 can be or include a semantic segmentation algorithm (e.g., an image segmentation algorithm), point matching algorithm (e.g., such as ICP), object detectors, computer vision models, labeling models, and/or any other suitable model. In a first example, the state determination model 130 can detect a target structure in the measurement and extract features and feature states (e.g., position, pose, trajectory, etc.) for the detected target structure. In a second example, the state determination model 130 can extract feature states for all structures depicted. In a third example, the state determination model 130 can detect features of a structure in the measurement and match them to a features of a virtual structure in a 3D representation of the subject 200. In this example, the virtual structure pose and/or shape can be adjusted based on feature states for features corresponding to the same structure as the virtual structure and/or feature states for nearby structures. In this example, the state determination model 130 can include an alignment model and/or can operate on the output of an alignment model. In a specific example, the method can include determining a trajectory for a structure and/or features thereof based on the measurements 300 thereof and/or feature states thereof (e.g., based on a timeseries of past states). In an example, determining the trajectory can include determining the path, an equation defining the path, and/or other variables (e.g., speed, acceleration, etc.) defining the motion of the structure and/or features thereof through time. The system can include one or more state determination models 130. In examples, a state determination model 130 can be specific to a target structure type (e.g., a particular medical instrument), can be specific to a feature, can identify multiple target structures and corresponding feature state values, can identify multiple features and corresponding features state values, and/or can be otherwise be organized.

However, the state determination model 130 can otherwise be configured.

The optional state prediction model 140 (e.g., example shown in FIG. 2A) functions to predict one or more future feature states. The state prediction model 140 can predict the future feature states based on: a current procedure state (e.g., a set of feature states) and/or the metadata thereof (e.g., timestep, step identifier, etc.), a set of prior procedure states (e.g., for the same or different procedure step) and/or the metadata thereof, current procedure step, a set of prior procedure steps, a step goal, a procedure goal, a procedure style cluster, a time series of feature states (e.g., output from S300), a set of measurements 300, and/or based on any other suitable input. The inputs to the state prediction model 140 (e.g., the vector values) can be of a constant or variable size. In a specific example, the size of the input depends on the duration of the procedure. The outputs of the state prediction model 140 can include the state of the procedure (e.g., a set of feature states) at one or more future timepoints, a next procedure step, and/or any other suitable information about a next procedure state. The state prediction model 140 can be: specific to the procedure, specific to the procedure step, specific to a procedural style (e.g., specific to the cluster), specific to a provider 10, specific to a target structure, generic for one or more of the above, and/or otherwise generic or specific. The output of the state prediction model 140 can be a single state prediction or a set of multiple state predictions. In a first variant of multiple state predictions, the multiple state predictions can be a set of candidate procedure states (e.g., wherein a provider 10 can select a procedure state from the set of candidate procedure states). In a second variant of multiple state predictions, each state prediction can be a prediction in state of a different structure associated with the subject 20 (e.g., a first state prediction can be a change in pose of a syringe needle and a second state prediction can be a change in pose of a syringe plunger). In a third variant of multiple state predictions, the set of state predictions can be an ordered list of state predictions. However, state predictions can be otherwise numbered and/or ordered. Each state prediction can include a change in pose of a structure (e.g., a set of vectors representing pose changes of different parts of a structure), a trajectory of a structure, an indication of a target structure and/or target feature to be operated on, a set of operations, a set of implements to be used in a next procedure step, a set of instructions (e.g., machine-readable code, a set of text instructions for a provider 10, an AR/VR UI element indicating information about the next procedure step), a 3D representation of the subject 200 in the next state, and/or any other suitable form of state prediction. Each state prediction can be defined relative to a virtual structure and/or virtual features, relative to an observed structure and/or observed features, relative to the world, relative to a provider 10, relative to a camera position, and/or relative to any other suitable system component. In a first variant, the state prediction (e.g., determined feature state) is relative to a virtual structure and/or feature of a virtual structure (e.g., the determined state of a medical tool can be relative to an internal subject structure of the subject 20), wherein the virtual structure is not represented in the measurements 300. In a second variant, the state prediction is relative to structures observed in the measurements 300 and/or observed features. The state prediction can be relative to a target structure (e.g., a tumor to be removed), a subject's surface (e.g., skin), a feature on a subject's surface (e.g., skin), a surgical implement, the hand of a provider 10, and/or any other suitable structure and/or feature thereof. However, the state prediction can be otherwise defined. Each state prediction can be within a world coordinate system, within a coordinate system defined by structures on the subject 20, within a coordinate system defined by observed or virtual features of the subject 20, and/or within any other suitable coordinate system.

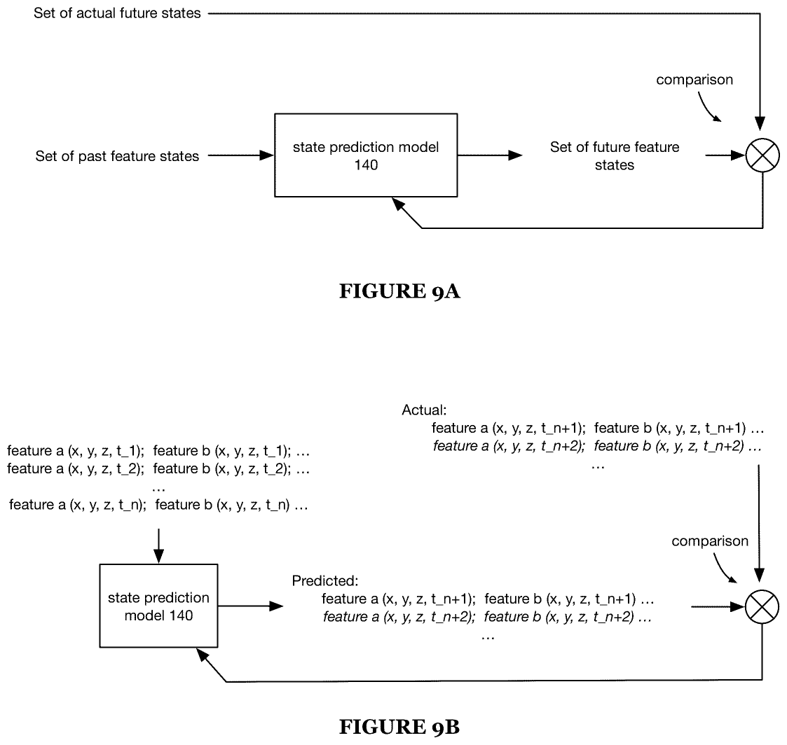

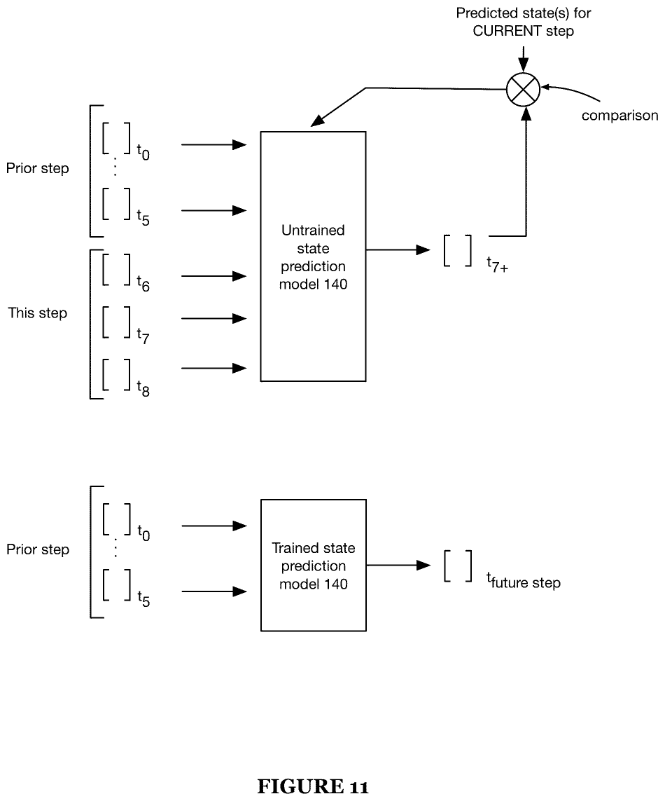

Training the state prediction model 140 can include using labeled, standardized training procedure data containing a time series of feature states of a training procedure for a set of measurements 300 (e.g., spanning a time interval preceding the a set of measurements containing the training target, 1 second, 5 seconds, 10 seconds, 10 frames, 20 frames, etc.; example shown in FIG. 9A and FIG. 9B). State prediction training targets can include a set of feature states of the training procedure at subsequent time point or a subsequent time interval (e.g., directly following the time interval, for the same procedure step, for the next procedure step, etc.). Training the state prediction model 140 can include predicting a state prediction, comparing it to the state prediction training target, and optionally updating the model (e.g., using backpropagation, etc.; example shown in FIG. 11). In a specific example, the state prediction model 140 can be trained by using structure feature states (and/or trajectories thereof) determined during S200 and/or S300 as training targets, and/or by using the structure feature states (and/or trajectories thereof) to determine the training targets (e.g., by averaging). However, the state prediction model 140 can otherwise be trained.

However, the state prediction model 140 can be otherwise defined.

Measurements 300, features, feature states, and/or training procedure data (e.g., referred to herein collectively as “structure data”) can optionally be standardized (e.g., registered, scaled, etc.), which functions to transform the structure data to a coordinate system shared across different sets of structure data from the same procedure or different procedures. Standardization can be performed before, after, or during any suitable step. Standardization can be performed on measurements 300, features, feature states, training procedure data and/or any other structure data. The coordinate system can include an origin; a set of axes using a cartesian, polar, cylindrical, spherical, geographical, curvilinear, and/or any other suitable type of coordinate system; and/or any other suitable coordinate reference frame elements. Standardization can include aligning the coordinate systems of different sets of structure data (e.g., sets of structure data from different measurements 300 in a time series, sets of structure data from different measurements 300 from different sensors at the same time step, sets of structure data from different procedure instances, and/or aligning the structure data sets to each other. Standardization can be performed for a procedure, for individual steps within the procedure, and/or for any other suitable procedure parts. In variants, data can be standardized (e.g., registered, scaled): based on a transformation calculated by matching keypoints between different data corpuses; using a calibration; using a known transform; and/or otherwise standardized. However, the structure data can be otherwise standardized.

However, the system can include any other elements, or be otherwise configured.

### 4. Method

As shown in FIG. 1, the method can include: determining measurements of a procedure S100, determining a set of feature states S200, tracking the set of features S300, optionally determining a procedure step S400, optionally predicting a set of future feature states S500, optionally performing further analyses based on the set of feature states S600, and/or any other suitable steps. However, the method can be otherwise performed.

The method is preferably performed using the system described above, but can alternatively be performed using any other suitable system.

All or portions of the method can be performed once (e.g., for one or more procedures, etc.), repeated, iteratively performed (e.g., for each of a set of procedures, steps, or subjects 20, etc.), responsive to occurrence of an event (e.g., responsive to receipt of a request), periodically, and/or at any other suitable time. All or portions of the method can be performed in real time, contemporaneously, asynchronously, in parallel, serially, and/or with any other suitable relationship. All or portions of the method can be performed automatically, manually, semi-automatically, and/or otherwise performed.

### 4.1 Determining Measurements of a Procedure S100.

Determining measurements of a procedure S100 functions to obtain a set of measurements 300 to use as input for other steps of the method. S100 can be performed using a sensor, received by the system (e.g., by the processing system of a computing device of the system) from an external source (e.g., a sensor of an external device, and/or otherwise performed. Measurement determination can be performed at any suitable time. In a first variant, measurements 300 can be sampled continuously throughout a procedure. In a second variant, a set of measurements 300 can be received after a procedure has taken place. However, S100 can be performed at any other suitable time.

In a variant, the relevance of each measurement 300 can be determined based on the procedure type, procedure step measurement, features and/or feature states determined from the measurement, and/or any other suitable input. Relevance can be: relevance to a current procedure step, relevance to a current procedure, and/or relevance to any other suitable attribute of a surgical procedure. Relevance can be determined based on measurements 300, feature states, procedure states, feature tracks, and/or any other suitable information. Relevance can be determined based on information relating to a medical tool (e.g., a medical instrument), provider structure, patient structure, physical structure, virtual structure, and/or any other suitable system component. Relevance can be determined using a trained model (e.g., neural network, etc.; trained using frames or clips of a procedure manually labeled for relevance, etc.); based on a set of heuristics (e.g., based on whether a set of predetermined objects are detected within the measurement, whether the set of predetermined objects are not detected within the measurement, etc.); based on whether a user instruction has been received (e.g., “not relevant”, “stop recording”, “start recording”, etc.); and/or otherwise determined. Irrelevant measurements can be ignored, downweighted, discarded, or otherwise managed. In an example, when a measurement 300 is deemed to be “irrelevant” (e.g., it depicts a provider 10 performing a non-surgical task; for example: pausing, cleaning a tool, changing gloves, checking a measurement, etc.) some or all of the remainder of the steps can be not performed until measurements 300 are determined to be relevant again. In this example, measurement relevance can be manually or automatically determined. When the relevance of a measurement is automatically determined, relevance can be determined using the step classification model 110 (e.g., where the determined step has a low confidence, where the determined step is “no step”, etc.), using the clustering model 120 (e.g., where a cluster for the set of measurements 300 and/or feature states determined from the set of measurements 300 is not found), using a set of heuristics (e.g., where a set of features associated with the provider features are over a threshold distance away from a target virtual structure, target subject structure, etc.), and/or any other suitable method. Additionally or alternatively, relevance determination can be performed using any of the methods used to segment training procedure data. Relevance can be determined at S100 but can alternatively be determined after the feature states are determined and/or tracked (e.g., in S200 and/or S300) during S600 (e.g., when deciding which feature states and/or measurements 300 to store for training a future state prediction model 140), and/or at any other suitable time. In a specific example, S500 is performed responsive to a set of feature states and/or time series of measurements 300 being relevant and is not performed otherwise. In a second specific example, S600 is performed responsive to a set of feature states and/or time series of measurements 300 being relevant and is not performed otherwise. However, measurement relevancy can be otherwise used.

However, determining measurements of a procedure S100 can be otherwise performed.

### 4.2 Determining a Set of Feature States S200.

Determining a set of feature states S200 functions to determine a set of feature states for a given structure (e.g., provider 10, instrument, etc.) based on the set of measurements 300. Determining a set of feature states preferably includes identifying features S110 and determining feature states for the identified features S220. S200 is preferably performed by the state determination model 130 but can additionally or alternatively be performed by any other suitable system component. In variants where the system includes multiple state determination models 130, S200 can optionally include selecting a state determination model 130 based on a determined procedure step (e.g., example shown in FIG. 2B).

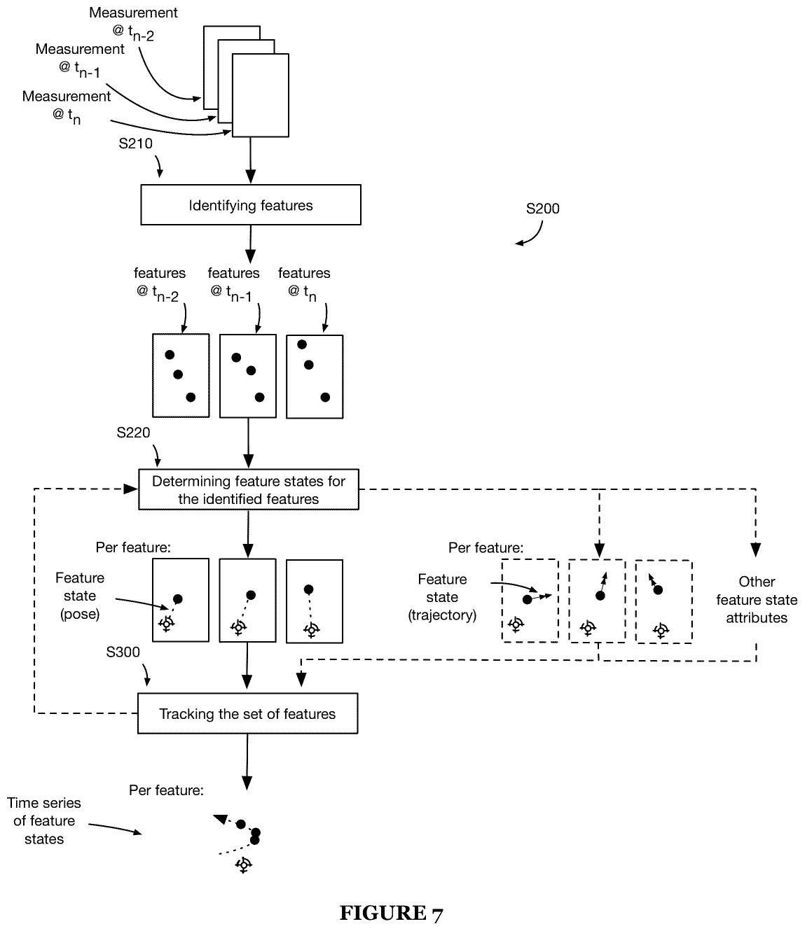

Identifying features S210 functions to determine features within the measurement 300 (e.g., observed features; example shown in FIG. 7) and/or a 3D representation of a subject 200 (e.g., virtual features). Features can be determined per-measurement (e.g., per frame, for camera-based methods) and/or can be determined using information from multiple frames captured in sequence. Each feature can be depicted in the set of measurements 300 (e.g., observed features) or not depicted in the set of measurements 300 (e.g., virtual features). Features can be represented in the 3D representation 200 (e.g., by virtual structures within the 3D representation 200) or not represented in the 3D representation 200. Features can be identified based on identified features from a prior timestep (e.g., from a previous frame) and/or can be identified without information about prior features. Virtual features can be extracted from the 3D representation 200 or otherwise determined (e.g., calculated) from the virtual structures within the 3D representation 200.

In a first variant, identified features can be observed features determined directly based on measurements 300. In this variant, features can be identified using semantic segmentation, instance segmentation, bounding box detection, keypoint detection, corner detection, edge detection, blob detection, texture analysis feature descriptors (e.g., SIFT, SURF, etc.), deep learning-based features (e.g., features output from intermediate and/or final layers of a neural network, intermediate embeddings output by a set of hidden layers, convolutional layers, pooling layers, and/or other layers, etc.), and/or other methods for determining features. In examples, identifying features can include: segmenting the measurement 300 (e.g., using semantic segmentation), labeling all components of a measurement 300 (e.g., each pixel within an image or video frame), classifying components of the measurement 300 (e.g., classifying a structure within the measurement), determining one or more features of one or more of the structures (e.g., identifying the position of each of a set of points, regions, bounding boxes, etc. associated with the structure within an image or video frame), and/or otherwise extracting information from the measurement.

In a second variant, identified features can be virtual features identified from a set of virtual structures (e.g., elements of a 3D representation 200 of a set of structures) registered or not registered with the set of measurements 300. In this variant, identifying features can include identifying a subset of virtual features which do not appear in the set of measurements 300, identifying a subset of virtual features which do appear in the set of measurements 300, identifying a subset of virtual features which were identified at a previous timestep (e.g., observed or virtual features identified in the previous timestep but not appearing in the set of measurements 300 at the current timestep, etc.), identifying a subset of virtual features which correspond to a current procedure step (e.g., features of relevant anatomical parts), and/or identifying a subset of virtual features selected on any other basis. In this variant, determining virtual feature states (e.g., S320) can be performed by registering the 3D representation 200 with the set of measurements 300. However, the identified features can be any other suitable type of feature.

In an example, a set of observed features are determined using the steps described in the first variant and a set of virtual features are determined using the steps described in the second variant. However, the variants can otherwise be combined.

However, S210 can be otherwise performed.

Determining feature states for the identified features S220 functions to determine state information of each identified feature (e.g., example shown in FIG. 7). S220 can be performed concurrently with S210 (e.g., wherein feature positions and/or orientations are determined as a part of feature identification) or after S210. S220 can be performed concurrently with or after S300 (e.g., wherein feature positions are estimated based on feature trajectories) or before S300. Determining feature states can include determining the pose, trajectory, alignment, and/or any other suitable feature parameter for a feature. The feature states can be determined in a predetermined coordinate system. The coordinate system can be a world coordinate system, a subject coordinate system, a structure coordinate system, a coordinate system of the 3D representation of the subject 200, and/or any other suitable coordinate system. During a registration process, the coordinate system can be aligned with a world coordinate system, a subject coordinate system, a structure coordinate system, a coordinate system of the 3D representation of the subject 200, and/or any other suitable coordinate system. In a specific example, feature states are tracked within a coordinate system of a 3D representation of the subject 200. In a second specific example, feature states are determined relative to one or more virtual structures of the 3D representation of the subject 200 (e.g., relative to an internal structure, such as a tumor or brain). Determining feature states can optionally include standardization. In a first variant, determining feature states can include standardizing the set of measurements 300 to a predetermined coordinate system (e.g., a reference frame defined by the 3D representation of the subject 200). In a second variant, determining feature states can include standardizing the determined feature states to a predetermined coordinate system. However, S220 can otherwise include standardization at any other suitable time.

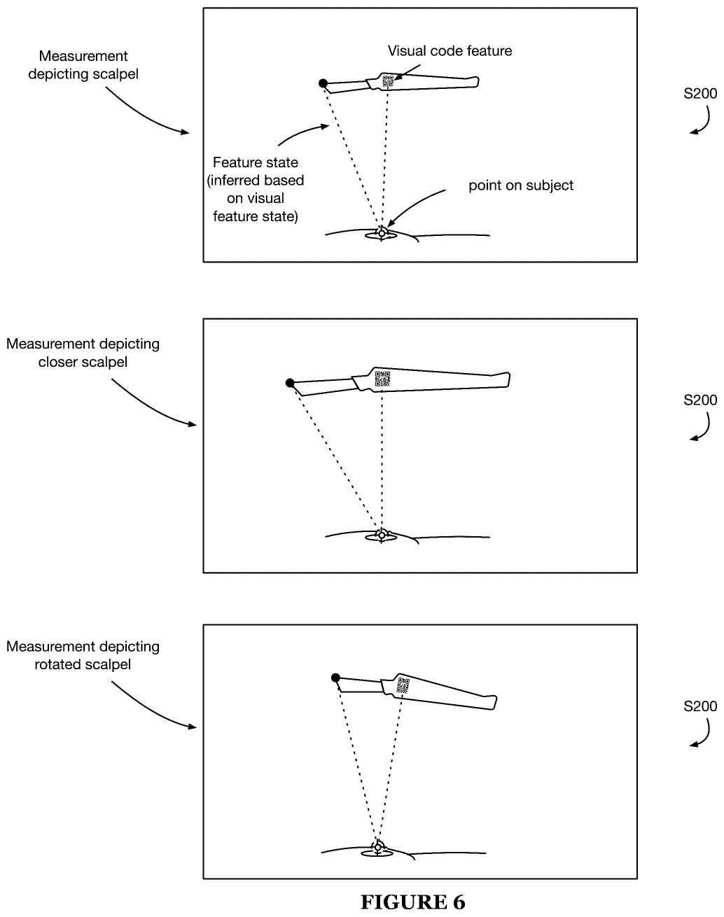

Determining feature states can include determining feature states for observed features and/or determining feature states for virtual features. In a first variant, determining feature states is performed for observed features. In this variant, determining feature states can include using visual odometry, visual-inertial odometry, stereo-based methods, structure from motion methods, monocular depth estimation, determination (e.g., extrapolation) of a feature position based on a known a feature trajectory, learning-based methods, Simultaneous Localization and Mapping (SLAM) methods, multi-view geometry, photogrammetry, hidden Markov models, and/or any other suitable method. In a first example, feature states for a set of observed features can be determined from a set of measurements alone. In a second example, feature states for observed features can be determined by combining the set of measurements 300 with a trajectory determined from a set of measurements 300 captured in a prior timestep and a feature state determined in the prior timestep (e.g., wherein an extrapolation provides an estimate of a feature state, and the measurement 300 is used to refine the estimate). In a third example, feature state for observed features can be determined by identifying a visual code (e.g., a QR code), estimating the state (e.g., pose) of the visual code, and optionally determining the feature state for other observed features with a known pose relative to the feature code based on the feature state for the visual code (e.g., example shown in FIG. 6). However, determining feature states for observed features can be otherwise performed.

In a second variant, determining feature states can be include determining feature states for virtual features. In examples, this can include: registering a 3D representation 200 (e.g., a predetermined virtual model generated ex situ from a set of scans; subject model modeling internal subject layers, etc.) with the set of measurements 300; and tracking the state (e.g., pose) of the 3D representation 200 and/or features thereof relative to the set of measurements 300, the medical structures, and/or another reference point. The 3D representation state can be tracked by: transforming the 3D representation pose based on a predetermined registration; iteratively reregistering the 3D representation 200 and measurements 300; constraining the alignment between the 3D representation 200 and the subject depiction in the measurement 300 using the registration (and/or alignment between the model features and measurement features), and/or otherwise maintaining the 3D representation-measurement alignment. The 3D representation 200 is preferably not constrained by a bounding volume drawn relative to the subject 20 detected in the measurement, but can alternatively be constrained by a bounding volume (e.g., defined by reference points determined within the measurements 300, such as subject extremities, joints, features, etc.). Determining feature states for virtual features can include using an alignment model to align the 3D representation 200 (e.g., and virtual features defined thereby) to the set of measurements 300 using an alignment model and a set of alignment features (e.g., a subset of the observed features). Aligning the 3D representation 200 can include aligning the entire 3D representation 200 to the set of measurements 300 or aligning a part of the 3D representation 200 to the set of measurements 300. The method can optionally include aligning different parts of the 3D representation 200 to the set of measurements 300 independently of each other or non-independently of each other. In a specific example, two parts of the 3D representation 200 separated by a joint are aligned independently of each other. In a second specific example, two parts of the 3D representation 200 separated by a joint are aligned with a point constraint on each alignment, the point constraint associated with the joint. This variant preferably uses feature states for observed features determined using methods described in the first variant. The alignment model functions to align the 3D representation 200 with the set of measurements 300. The alignment model can leverage: point set registration (e.g., a point matching algorithm, iterative closest point, such as ICP, N-ICP, etc.; robust point matching; etc.), which registers matching features of virtual structures of the 3D representation 200 (e.g., an exterior structure representation) and alignment features; a spatial mapping algorithm; mesh alignment methods; correspondence-based registration; and/or any other suitable algorithm. In embodiments where the 3D representation 200 is displayed on a VR/AR headset (e.g., overlaid over a set of measurements 300, overlaid over a lens, overlaid over a display, etc.), the alignment model can additionally use measurements 300 captured by an accelerometer and/or any other motion tracking system to keep the 3D representation 200 aligned with the subject 20 (e.g., the subject 20 depicted in the set of measurements 300 or the subject 20 viewed through the lens). Alignment features can be used to indicate points or regions of interest for a particular application. Preferably, alignment features are keypoints, but can alternatively be any other suitable type of feature. Preferably, alignment features are a subset of a set of observed features (e.g., observed features which represent a subject 20; as opposed to a medical tool), but alignment features can alternatively be any other suitable type of feature. Alignment features can be tracked and used for future state prediction, or untracked (e.g., used purely for registration, etc.). Alignment features can each be associated with a virtual feature on an interior and/or exterior structure (e.g., external features). In examples, alignment features can indicate the location of: a right ear, a left ear, a mouth, a nose, a forehead (e.g., center of the forehead), a center of mass, an abnormal growth, a surgical incision point (e.g., Kocher points), and/or any other point of interest. Preferably, alignment features are represented by a set of 3D coordinates, but can alternatively be represented by a set of 2D coordinates, and/or otherwise represented. In an example, an alignment feature can be an observed feature on the surface of the subject 20, and the positions of the virtual structures within the set of virtual structures can be determined based on their estimated relative position to a virtual feature corresponding to the observed feature (e.g., the alignment feature), a coordinate reference frame aligned with the observed feature (e.g., via registration), a virtual structure (e.g., of the 3D representation 200), and/or relative to another reference. The states (e.g., poses) of virtual features can be determined based on the states of alignment features (e.g., observed features). In a first example, a set of states of target virtual features of a target structure can be determined based on an image depicting a subject exterior by registering the image with a 3D representation of the subject 200 (e.g., the external structure represented within the 3D representation) using the positions of the set of target virtual features relative to the alignment features. In a second example, a set of features for a surgical structure (e.g., an implant) can be determined by estimating the position of the surgical structure (e.g., a stent) using the 3D representation 200, a 3D representation of a surgical implement (e.g., a catheter) connected to the surgical structure, and a measurement 300 depicting a visible portion of the surgical implement. In this example, the surgical implement position can be estimated to follow the path of a virtual structure (e.g., an artery) within the 3D representation 200, and the surgical structure can be estimated to be near a feature of the surgical implement (e.g., a catheter tip). In a third example, when the position of a physical structure (e.g., an organ) is not aligned with the corresponding virtual structure, the virtual structure pose can be adjusted to align with the observed physical structure along with the corresponding features of the virtual structure. In a fourth example, when a physical structure is removed from a subject's body, the virtual structure corresponding to the physical structure can be removed from the 3D representation 200, and the corresponding features of the virtual structure can be additionally removed. In a fifth example, the pose of a virtual structure can be adjusted to account for real-world physics based on observations derived from the measurements 300 (e.g., the position of features on a pliant virtual structure can be translated to account for the estimated impact of gravity on the physical structure given the orientation of the subject 20). In a sixth example, a set of transforms between the measurement space (e.g., the current real-world space) and the 3D representation space can be calculated based on matched alignment features, wherein the 3D representation 200 can be transformed into the measurement space (and aligned with the measurement 300 depicting the subject 20) based on the set of transforms. Additionally or alternatively, the poses of virtual features can be estimated based on their trajectories during their most recent observation. In an example, the position of a feature observed in a previous timestep can be extrapolated based on the trajectory of the feature determined during the previous timestep.

However, determining feature states for the identified features S220 can be otherwise performed.

However, determining a set of feature states S200 can be otherwise performed.

### 4.3 Tracking the Set of Features S300.

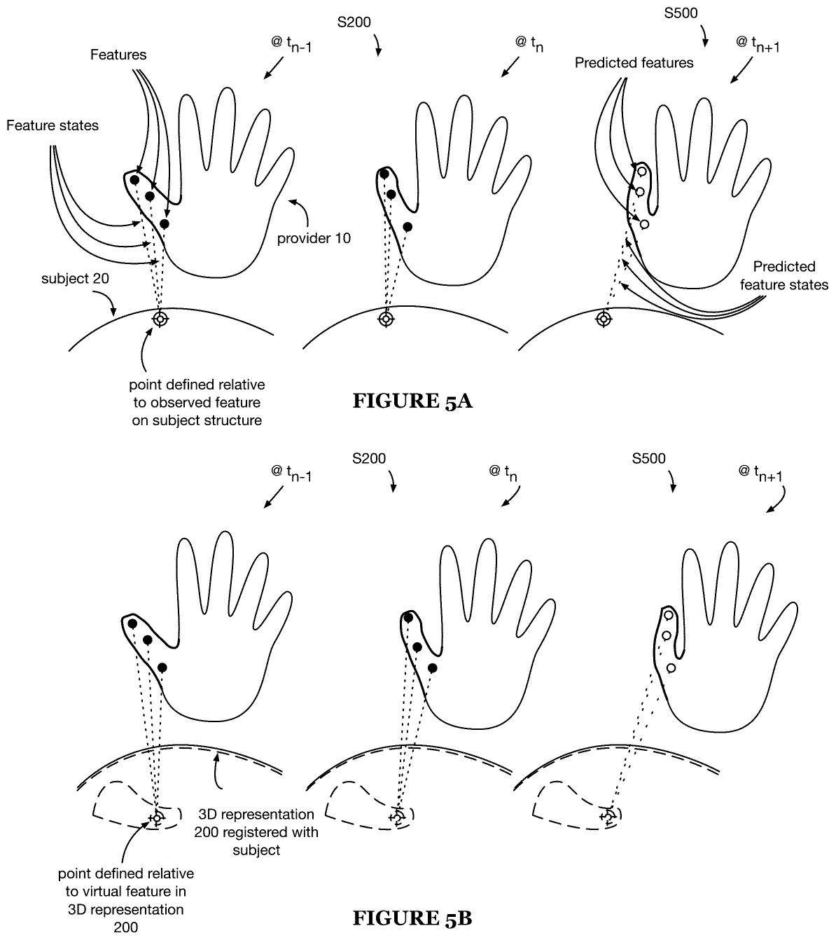

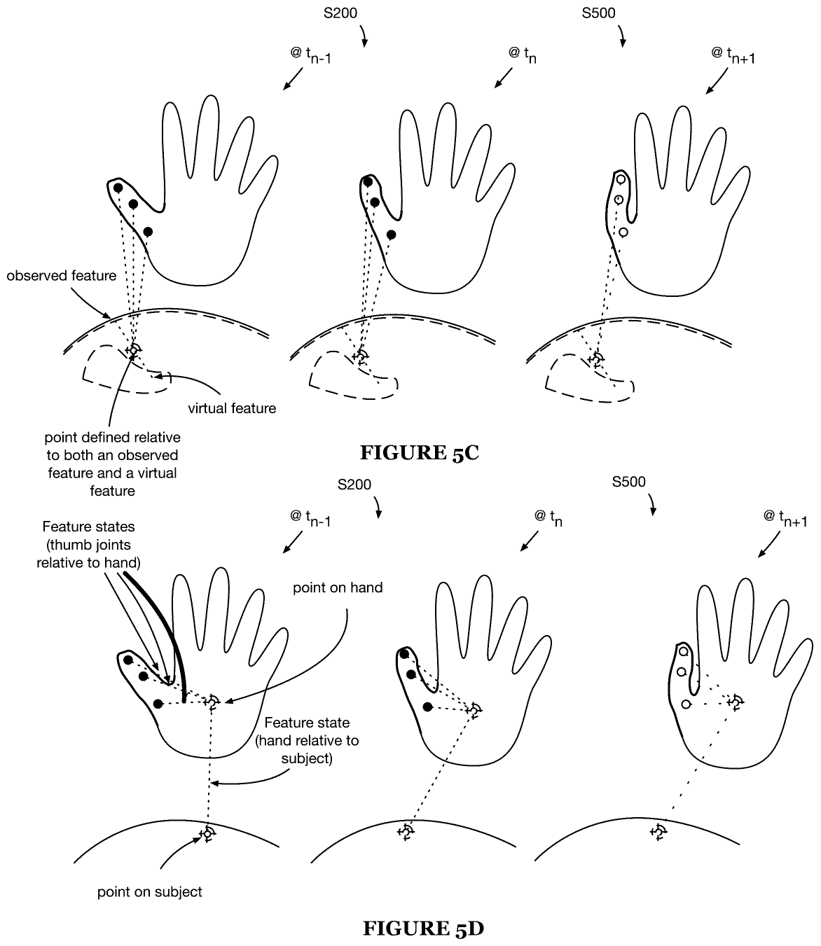

Tracking the set of features S300 functions to determine a time series of feature states, feature tracks, and/or other outputs. The features are preferably tracked through successive measurement frames, but can alternatively be tracked in another domain. Tracking the set of features can include matching features determined from measurements 300 captured in successive timesteps but can alternatively include matching features determined in any other suitable pair of timesteps. The output of tracking the set of features can be a time series of feature states (e.g., trajectories, poses, feature tracks, etc.) for each feature (e.g., example shown in FIG. 7) but can alternatively include any other suitable output. Tracking the set of features is preferably performed for features corresponding to a medical tool (e.g., a surgeon hand, a medical instrument, etc.) but can additionally or alternatively be performed for features corresponding to other provider structures, subject structure, and/or other features. Tracking the set of features can rely on: simultaneous localization and mapping (SLAM), a state estimation algorithm, odometry methods, optical flow methods, Kalman filtering, object tracking methods, localization methods, iterative model alignment or matching, and/or any other suitable algorithms. In a first variant, S300 can include matching features based on the positions of each feature in their respective measurement. In a second variant, S300 can include matching features based on the feature state of each feature. In a third variant, S300 can include matching features based on their classification (e.g., determined via a segmentation step performed on the corresponding measurement). In a fourth variant, S300 includes predicting the feature state for a current timestep using the feature state for a feature from a prior timestep (prior feature), then matching the prior feature with a feature from the current timestep (current feature) when the predicted feature state substantially matches the current feature's feature state. However, features can be otherwise matched across timesteps. Matching can include matching observed and/or virtual features determined for a prior timestep with observed and/or virtual features determined for a current timestep. In an example, responsive to an observed feature in a prior measurement becoming occluded in a current measurement, a corresponding virtual feature with an inferred state (e.g., inferred in S220) close to the state of the observed feature can be matched to the observed feature. In a second example, a newly-observed feature (e.g., a feature which appears in a current measurement but not a prior measurement) can be matched with a corresponding virtual feature with an inferred state at the prior timestep. In a first variant, all features are tracked in the same coordinate system. In a second variant, features for different structures are tracked in different coordinate systems. In an example of this variant, a first set of features (e.g., provider features) is tracked relative to a second set of features (e.g., provider features and/or subject features); and the second set of features is tracked relative to a third set of features (e.g., subject features; example shown in FIG. 5D). Features can be tracked within the same or different coordinate system as the coordinate system in which the features are determined. Examples of feature systems in which features can be tracked include coordinate systems defined by observed features (e.g., example shown in FIG. 5A), coordinate systems defined by virtual features and/or a 3D representation of the subject 200 (e.g., example shown in FIG. 5B), coordinate systems defined by both observed and virtual features (e.g., example shown in FIG. 5C), and/or any other coordinate system defined by any other suitable features. However, one or more coordinate systems can otherwise be used.

However, tracking the set of features S300 can otherwise be performed.

### 4.4 Determining a Procedure Step S400.

The method can optionally include determining a procedure step S400 functions to determine the step of the procedure at a timestep (e.g., a current timestep, a past timestep, etc.). S400 functions to reduce the amount of data needed to predict the next state of the procedure in S500, to train the model in S100, and/or can be otherwise used. The procedure step can be determined based on a previous procedure step or can be determined without knowledge of the previous procedure step.

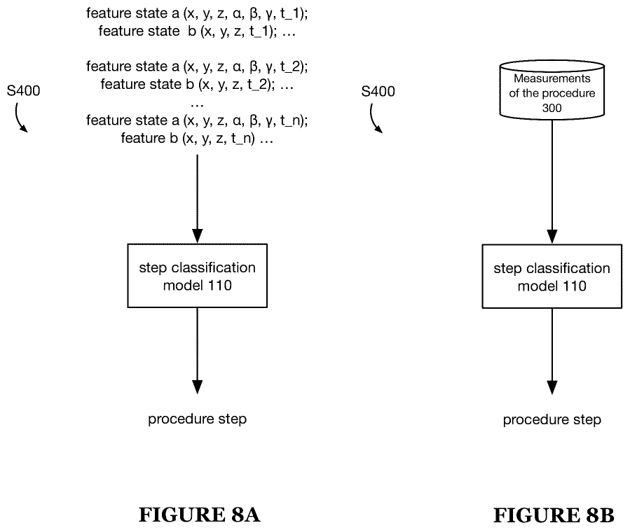

In a first variant, the step is determined automatically based on measurements 300 (e.g., labeled or unlabeled) using the step classification model 110. In this variant, the step can be determined directly based on the measurements 300 (e.g., example shown in FIG. 8B), based on a set of feature states for the measurements 300, based on a time series of feature states (e.g., feature tracks, tracked features, etc.) (e.g., example shown in FIG. 8A), and/or based on any other suitable information. In a first example, the step is determined based on a set of feature states corresponding to provider features (e.g., “non-patient features”) at one or more timesteps. Preferably, the feature states are feature states dynamically extracted from the most recent set of measurements within the time series of measurements 300 but can alternatively be feature states extracted from any other suitable set or subset of measurements within the time series of measurements 300. In a second example, the step is determined based on a set of observed features representing a set of subject structures and/or virtual features representing a set of virtual structures (e.g., corresponding to subject structures). In a specific example, the step can be determined based on the relative states of features representing subject structures and features representing provider structures. However, step classification can be otherwise automatically performed based on measurements 300.

In a second variant, the method can include receiving the step as specified by a user (e.g., the provider 10). In variants, the user can specify the step (e.g., with a voice command, by making a selection on a computer, etc.). The user can specify the step during the procedure (e.g., wherein the user is performing the procedure, wherein the user is observing the procedure, etc.), after the procedure (e.g., the user can specify the timeframe during which a step was being performed), and/or at any other suitable time. In a specific example, the user can specify the step with a voice command (e.g., by narrating steps of the procedure) to an audio recording device of the system. In a second specific example, the user can specify the step on a computer by typing, selecting a choice from a list, checking off the step in a procedure checklist, and/or otherwise indicating the step.

However determining a procedure step can be otherwise performed.

### 4.5 Predicting a Set of Future Feature States S500.

Optionally predicting a set of future feature states S500 functions to determine a state prediction (e.g., a set of future feature states) at one or more future timesteps given a set of states (e.g., a time series of feature states) of the procedure. S500 can optionally include determining a procedure step and/or procedural style (e.g., using the clustering model 120 and/or step classification model 110) and predicting a set of future feature states based on the determined procedure step and/or procedural style. The state prediction can be used in S600 for downstream analyses. S500 is preferably performed using the state prediction model 140 but can be otherwise performed by another system component. Inputs to the state prediction model 140 are preferably standardized to match the reference frame of training procedure data on which the state prediction model 140 was trained but can be otherwise standardized or not standardized. S500 can be performed in real time (e.g., during a procedure), continuously, iteratively, and/or otherwise performed. S500 can be performed on observed features (e.g., features appearing in the set of measurements 300) and/or virtual features (e.g., features of virtual structures within the 3D representation 200). S500 can be performed on all features or on a subset of features, wherein the subset of features is determined based on relevance of each feature to the procedure, relevance of each feature to the procedure step, structure type associated with each feature, trajectory of each feature, and/or any other suitable attribute. S500 can be performed using the state prediction model 140, a hidden markov model, a neural network trained to predict the trajectory for a predetermined number of future timesteps (e.g., using backpropagation, rewards, etc.), and/or using any other suitable model.

In a first variant, S500 includes determining a state prediction for one or more timesteps, based on the time series of feature states. The timestep can be the next timestep, a subsequent timestep, multiple future timesteps (e.g., the next future timesteps), and/or any other set of timesteps. In examples, the future timesteps can include a specific time interval (e.g., the next second, the next 10 seconds, etc.), a specific quantity of timesteps (e.g., the next 50 timesteps), all timesteps until the end of the step, all timesteps until the end of the procedure, and/or be otherwise defined. The time interval and/or quantity of future timesteps can be: predetermined (e.g., a fixed value), variable (e.g., dependent on the procedure, step, state, measurement set, etc.), input as a parameter to the state prediction model 140, dynamically determined based on the procedure and/or step, and/or otherwise specified.

In a second variant, S500 includes predicting a trajectory for one or more features for the procedure, based on the time series of feature states. This can include predicting a discrete set of future procedure states, a continuous approximation of future procedure states, and/or otherwise predicting the trajectory. In examples of this variant, an output of S500 for a feature can be a trajectory and/or a pose at a future timestep determined based on the trajectory.

However predicting a set of future feature states can be otherwise performed.

### 4.5 Performing Further Analyses Based on the Set of Feature States S600.

The method can optionally include performing further analyses based on the set of feature states S600, which functions to apply the prediction of S500 and/or the time series of feature states to an additional output. S600 can be performed during a procedure or after a procedure. Optionally, S600 can include displaying the predicted next state during the procedure (e.g., to the provider 10) on a display (e.g., a monitor, a screen, a headset, etc.). S600 can be performed by an analysis module 400 running on a local or remote computing system; however, S600 can be performed aby another suitable entity. Variants of S600 can include using a target trajectory (e.g., optimal trajectory) or set of target trajectories (e.g., optimal trajectories) which represent generalized target motion of features (e.g., states) during a procedure step (e.g., spanning multiple successive timesteps in a time window). The target trajectory can be manually-determined or automatically-determined. In a variant where the target trajectory is manually-determined, the target trajectory can be drawn within a virtual space (e.g., augmented reality space, virtual reality space, etc.) aligned with the measurement space and/or with the 3D representation 200. In a first variant where the target trajectory is automatically-determined, the target trajectory can be generated from an average of multiple time series of feature states, each from a different iteration of a particular procedure step of a prior iteration of a procedure. Optionally, the multiple time series can share a same procedural style. In a second variant where the target trajectory is automatically-determined, the target trajectory can be a trajectory from a prior procedure with a high procedure score (e.g., determined in the second variant of S600) or an aggregation of trajectories with high procedure scores from different procedures (e.g., determined using a trajectory model). The target trajectory is preferably an absolute target trajectory but can alternatively be a relative target trajectory (e.g., relative to a feature of the 3D representation 200, relative to a feature of an inner subject layer, etc.), a target trajectory given a recent time series of feature states for a procedure. The target trajectory can be defined relative to observed features, virtual features, subject structures, non-subject structures (e.g., provider structures), virtual structures, and/or any other suitable element or combination of elements. In a specific example, the target trajectory represents the path of a tool interacting with a subject structure (e.g., a scalpel tip path across a portion of skin to be cut). However, the target trajectory can be otherwise determined. In variants where the predicted next state and/or target trajectory is displayed, the method can additionally include using a tracking algorithm to correct for changes in position of a camera.

In a first variant, S600 can include training a robot to perform a procedure and/or guiding the robot to perform the procedure. The robot can be trained on a per-step basis, on a per-procedure basis, trained to learn based on analysis conducted for a plurality of cases, trained to mimic a specific case, and/or otherwise trained. Training the robot can include training the state prediction model 140 and using the output of the state prediction model 140 to generate robot commands. In an example, a robot is assigned the task of replicating a procedure and/or step. In a first embodiment, the robot can be trained to: determine a set of measurements 300 for a procedure performed by the robot (e.g., as in S100), determine and track a set of feature states (e.g., as in S200 and S300), optionally determine a procedure step (e.g., as in S400), predict a future feature state using the state prediction model 140 (e.g., as in S500), convert the future feature state into robot instructions (e.g., based on the current robot state), and control the robot based on the robot instructions (e.g., converting the motion to a robotic control space and sending the trajectory or other control sequence to the robot). In a second embodiment, training the robot can include: determining a set of robot motions using a separate robot control model, and training the robot control model against the trajectory that is output by the state prediction model 140. In a third embodiment, the robot can be trained to execute the target trajectory. However, the robot can be otherwise trained and/or guided.

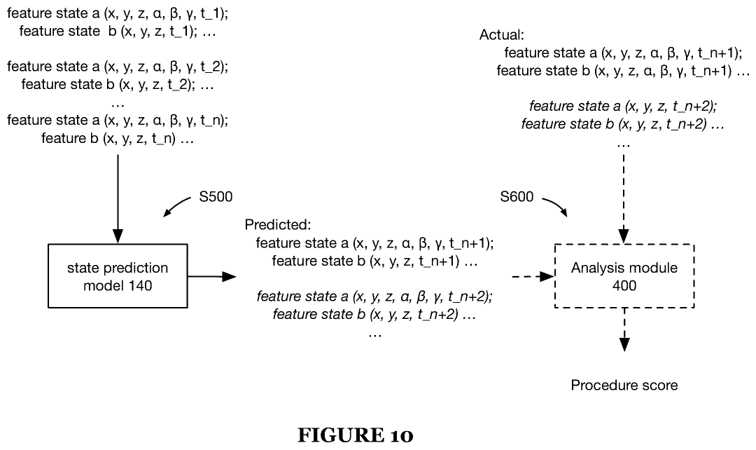

In a second variant, S600 can include evaluating performance of a provider 10 performing a procedure, which can include predicting the next state of the procedure S500, sampling a measurement of the actual next step of the procedure S200, determining a set of feature states at the next step of the procedure based on the measurement, and comparing the actual set of feature states to the predicted set of feature state (e.g., example shown in FIG. 2C). Alternatively, evaluating performance of the provider 10 performing a procedure can include comparing a time series of tracked feature states to a target trajectory. A procedure score can be determined based on the actual next state and predicted next state (e.g., a percent difference, a confidence score, etc.; example shown in FIG. 10). S600 can be performed during a procedure (e.g., giving live feedback to the provider 10) or after a procedure (e.g., to evaluate the performance of the procedure overall and/or individual steps within the procedure). Optionally, S600 can include displaying the procedure score (e.g., to the provider 10; example shown in FIG. 2C). Optionally, an aggregate procedure score can be determined by predicting the next state at many time points throughout the procedure, and comparing these with the actual next states. In an example of this variant, the procedure score can represent and/or include distance (e.g., cosine similarity, etc.) of an actual feature state and/or trajectory from a predicted feature state and/or trajectory. However, the provider performance can be otherwise evaluated.

In a third variant, S600 can include determining indicators of procedural success, which can include any techniques for model explainability (e.g., correlations, lift analysis, etc.). In examples, S600 can include determining a quality of a medical device (e.g., style, type, size, shape, texture, relative size or shape as compared to a provider hand, etc.) that is correlated with an effective procedural style and/or procedural outcome. In further examples, 600 can include determining a set of features (e.g., hand position, hand trajectory, etc.) that are more effective.

In a fourth variant, S600 can include guiding a provider 10. In this variant, trajectories (e.g., an optimal trajectory), suggestions, a manually-specified trajectory, and/or a procedure score (e.g., a procedure score determined in the second variant) can be displayed on a display visible to the provider 10. Suggestions can include suggested (e.g., predicted) surgical implement paths generated based on feature states for the surgical implement, text describing a next step, highlighted structures relating to a next step, and/or any other suitable form of suggestion. In an example, S600 can include determining whether the state of a feature (e.g., a provider feature) deviates from a target trajectory and displaying a notification to the provider 10 (e.g., including a trajectory deviation notification, instructions on how to resolve the deviation, etc.). In this example, deviation can be evaluated by using a distance (e.g., between the feature state and the target trajectory), an angle (e.g., between the trajectory of the feature and the target trajectory), and/or any other suitable metric. The display can be a monitor, an AR/VR headset worn by the provider 10, physical structure onto which information is displayed, and/or any other suitable form of display. However, a provider 10 can be otherwise guided.

In a fifth variant, S600 can include determining parameters for an external action, based on the medical structure track relative to the internal subject structure. This can include: determining the medical structure track relative to the (virtual) internal subject structure, predicting a target track based on the internal subject structure, and determining the external action parameters (e.g., where to make an incision, what external path or trajectory to use to target a point in the internal structure, etc.) based on the predicted target track (e.g., by extrapolating the track to the subject's exterior in measurement space or global coordinates, etc.). For example, because the medical structure (e.g., hand, instrument) state relative to the internal subject structure (e.g., organ, tumor) is directly tracked (e.g., instead of inferred), accurate medical procedure guidance can still be provided, even if the physical external layers of the subject shift. In an illustrative example, an organ can still be accurately targeted in a laparoscopic procedure even if the subject's fat or skin shifts.

However performing further analyses based on the set of feature states can be otherwise performed.

Different processes and/or elements discussed above can be performed and controlled by the same or different entities. In the latter variants, different subsystems can communicate via: APIs (e.g., using API requests and responses, API keys, etc.), requests, and/or other communication channels. Communications between systems can be encrypted (e.g., using symmetric or asymmetric keys), signed, and/or otherwise authenticated or authorized.

Alternative embodiments implement the above methods and/or processing modules in non-transitory computer-readable media, storing computer-readable instructions that, when executed by a processing system, cause the processing system to perform the method(s) discussed herein. The instructions can be executed by computer-executable components integrated with the computer-readable medium and/or processing system. The computer-readable medium may include any suitable computer readable media such as RAMs, ROMs, flash memory, EEPROMs, optical devices (CD or DVD), hard drives, floppy drives, non-transitory computer readable media, or any suitable device. The computer-executable component can include a computing system and/or processing system (e.g., including one or more collocated or distributed, remote or local processors) connected to the non-transitory computer-readable medium, such as CPUs, GPUs, TPUS, microprocessors, or ASICs, but the instructions can alternatively or additionally be executed by any suitable dedicated hardware device. Embodiments of the method can be performed on an application (e.g., a computer application, an application on an extended reality headset, etc.).

Embodiments of the system and/or method can include every combination and permutation of the various system components and the various method processes, wherein one or more instances of the method and/or processes described herein can be performed asynchronously (e.g., sequentially), concurrently (e.g., in parallel), or in any other suitable order by and/or using one or more instances of the systems, elements, and/or entities described herein.

As a person skilled in the art will recognize from the previous detailed description and from the figures and claims, modifications and changes can be made to the embodiments of the invention without departing from the scope of this invention defined in the following claims.

We claim:

1. A method for medical procedure tracking, comprising:
determining a virtual patient model comprising a virtual external patient structure and a virtual internal patient structure; and
during a medical procedure, performed using a set of medical instruments:
sampling a timeseries of measurements depicting a patient;
registering the virtual patient model with a measurement from the timeseries, using model features extracted from the virtual external patient structure and alignment features extracted from the measurement;
overlaying the virtual patient model over measurements of the timeseries based on the registration;
generating a set of instrument tracks by tracking the set of medical instruments relative to the virtual internal patient structure throughout the timeseries of measurements; and
predicting a future trajectory of the set of medical instruments relative to the internal patient structure based on the set of instrument tracks.

2. The method of claim 1, wherein the virtual external patient structure and the virtual internal patient structure are generated from the same set of cross-sectional patient scans.

3. The method of claim 1, further comprising identifying a current step in the medical procedure, wherein the future trajectory is predicted based on the identified current step.

4. The method of claim 3, wherein the current step is identified based on the set of instrument tracks.

5. The method of claim 4, further comprising evaluating whether the timeseries of measurements is relevant to the current step based on the set of instrument tracks, wherein the future trajectory is predicted responsive to the timeseries of measurements being relevant.

6. The method of claim 1, wherein registering the virtual patient model comprises aligning the virtual patient model and each measurement, virtual patient model registration is constrained by an alignment between the model features and the alignment features.

7. The method of claim 6, wherein the aligning the virtual patient model is performed using iterative closest point (ICP).

8. The method of claim 1, wherein generating the set of instrument tracks comprises:
detecting medical instruments from the set of medical instruments depicted within each measurement using a set of medical instrument object detectors trained to output a medical instrument detection and a kinematic state of the detected medical instrument;
matching the medical instruments across successive measurements; and
generating an instrument track for the medical instruments from the matches and the kinematic states.

9. The method of claim 1, wherein predicting the future trajectory of the medical tool feature comprises using a prediction model trained on trajectories of medical tool features determined during prior procedures.

10. A method, comprising:
determining a time series of images each depicting a patient and a non-patient structure;
determining a set of patient features representing the patient and a set of non-patient features representing the non-patient structure based on an image in the time series of images;
registering an internal model of the patient with the set of patient features;
for a non-patient feature in the set of non-patient features, tracking a position of the non-patient feature relative to the internal model; and
determining a predicted position of the non-patient feature relative to the internal model at a future timestep.

11. The method of claim 10, further comprising determining a current procedure step based on the position of the non-patient feature.

12. The method of claim 11, wherein the predicted position of the non-patient feature is additionally determined based on the current procedure step.

13. The method of claim 10, wherein the internal model comprises a 3D representation of a patient internal structure and is generated from a set of CT scans of the patient.

14. The method of claim 10, wherein the internal model is registered with the set of patient features by:
matching the set of patient features with a set of external features associated with the internal model; and
determining a pose of the internal model based on the match and a known relative position of the external features and internal model.

15. The method of claim 10, wherein the internal model comprises multiple parts and wherein each part is registered independently with the set of patient features.

16. The method of claim 10, wherein the internal model is rendered within an augmented reality display based on the registration.

17. The method of claim 10, further comprising determining whether the state of the medical tool feature deviates from a target trajectory defined relative to the internal model.

18. The method of claim 17, wherein the target trajectory is determined using a trajectory model, and wherein the method further comprises training the target trajectory model using the position of the non-patient feature.

19. The method of claim 10, further comprising determining instructions for a robot based on the predicted position of the non-patient feature.

20. The method of claim 10, wherein the medical tool feature is tracked relative to an internal structure of the patient represented within the internal model.
